(19) United States
(12) Patent Application Publication
(10) Pub. No.: US 2013/0315998 A1
(43) Pub. Date: Nov. 28, 2013

(54) Topical Foam Composition

(71) Applicant: CIPLA Limited, Mumbai (IN)

(72) Inventors: Amar Lulla, Mumbai (IN); Geena Malhotra, Mumbai (IN)

(73) Assignee: CIPLA Limited, Mumbai (IN)

(21) Appl. No.: 13/959,193

(22) Filed: Aug. 5, 2013

Related U.S. Application Data
(63) Continuation of application No. 13/511,301.

(30) Foreign Application Priority Data
Nov. 23, 2009 (IN) 2703/MUM/2009

(51) Int. Cl. A61K 9/12; A61K 31/439

(52) U.S. Cl. CPC A61K 9/122; A61K 31/439
USPC 424/489; 546/40; 514/279; 424/45; 514/161; 514/150; 514/171; 222/402.1

(57) ABSTRACT

A topical foam pharmaceutical composition for rectal administration comprising rifaximin is described. Also described is a method of making the composition and the use of the composition as a medicament.

### CROSS-REFERENCE TO RELATED CASES

This application is filed under 35 U.S.C. §111(a) as a continuation application which claims priority under 35 U.S.C. §119, 35 U.S.C. §120, and the Patent Cooperation Treaty to: parent application U.S. Ser. No. 13/511,301, filed under 35 U.S.C. §371 on May 22, 2012; which claims priority to PCT/GB2010/002164, filed under the authority of the Patent Cooperation Treaty on Nov. 23, 2010, published; which claims priority to Indian Application Ser. No. 2703/MUM/2009, filed Nov. 23, 2009.

### TECHNICAL FIELD

The present invention relates to a topical foam composition of rifaximin suitable for rectal administration, its process of manufacturing and its use for the treatment, prophylaxis, or maintenance of remission of colonic, anal or rectal dysfunction.

### BACKGROUND AND PRIOR ART

Anal disorders including anal fissure, anal ulcer, and acute haemorrhoidal disease and benign conditions of the anal canal, are common amongst the subjects of all ages, races and sexes. However, these conditions can be problematic to treat and inconvenient if not painful to endure. A subject with an anal fissure or ulcer frequently experiences anal pain and bleeding, the pain being more pronounced during and after bowel movements.

Haemorrhoids are specialized vascular areas lying subjacent the anal mucosa.

Various therapies have been devised to treat these anal disorders. Typical, non-surgical therapy includes bulk laxatives and sitz baths. Sitz baths are helpful because they induce relaxation of the anal sphincter mechanism. (Shafik, “Role of warm-water bath in anorectal conditions: The thermosphincteric reflex, “J. Clin. Gastroenterol., 16:304-308, 1993).

Topical anal therapy is also one of the approaches used in an effort to promote healing, relieve pain, and reduce swelling and inflammation. Many preparations have been tried including those containing local anesthetics, corticosteroids, astringents, antibiotics and other agents.

Although administration via the peroral route is the most commonly targeted goal of new drug and dosage form research and development, oral administration is not always feasible or desirable. The potential for oral dosage form development is severely limited for active agents that are poorly absorbed in the upper gastrointestinal (GI) tract and unstable to proteolytic enzymes. Some agents cause local stomach or upper GI irritation or require doses in excess of 500 mg. Certain patient populations, notably children, the elderly, and those with swallowing problems, are often difficult to treat with oral tablets and capsules. Additionally, treatment of some diseases is best achieved by direct administration near the affected area, particularly with diseases involving anorectal tissues. Although oral administration can be used for drugs targeted for some of these diseased tissues, exposure of the entire body compartment to the administered drug is inefficient and can lead to undesired adverse effects.

Rectal drug administration is amenable, however, to both local and systemic drug delivery. It has been effectively utilized to treat local diseases of the anorectal area as well as to deliver drugs systemically as an alternative to oral administration. Some advantages of this targeted delivery which includes large surface area, ability to bypass first-pass metabolism, prolonged residence time makes this route more promising for delivery of locally acting drugs.

Suppositories, solutions, suspensions, or retention enemas represent some of the rectal dosage forms. Of these, liquid preparations have very limited application, largely due to inconvenience of use and poor patient compliance. Semi-solid preparations like gels, foams or ointments for rectal administration can afford advantages over liquid formulations because retention of the dosage form in the rectal cavity reduces patient compliance problems.

However, none of the formulations available has been convincingly shown to reduce the healing time or to reliably ameliorate associated pain and some of the treatments, such as Neosporin® ointment (which contains three antibiotics Neomycin, Polymyxin B Sulfate and Bacitracin Zinc), are very sensitizing. In conclusion, antibiotics have not been found useful in treating the diseases. There was a need in the art to provide compositions useful to reduce healing time, alleviate pain and promote healing of the affected rectal and anal tissues.

Rifaximin is a water insoluble, semisynthetic rifamycin-based non-systemic antibiotic belonging to the rifamycin class of antibiotics, and has the scientific name [(2S,16Z,18E,20S,21S,22R,23R,24R,25S,26S,27S,28E)-5, 6,21,23,25-pentahydroxy-27-methoxy-2,4,11,16,20,22,24,26-octamethyl-2,7(epoxypentadeca-[1,11,13]trienimino)benzofuro [4,5-e]pyrido [1,2-a]-benzimida-zole-1,15(2H)-dione,25-acetate].

Rifaximin has been described to be endowed with an antibacterial activity similar to the activity of rifampin [Venturini A. P. and Marchi E., Chemiotherapia, 5 (4), 257-256, (1986)]. However, its mechanism of action differs from rifampin in that it is not absorbed through the systemic route after oral administration [Venturini A. P., Chemotherapy, 29, 1-3, (1983) and Cellai L. et al., Chemiotherapia, 3, (6), 373-377, (1984)] due to the zwitterionic nature of the compound, which cannot be absorbed by the gastrointestinal tract [Marchi E. et al., J. Med. Chem., 28, 960-963, (1985)].

Rifaximin is soluble in alcohol, ethyl acetate, chloroform and toluene. It exerts its broad-spectrum antibacterial activity by inhibiting bacterial RNA synthesis in the gastrointestinal tract against localized gastrointestinal bacteria that cause infectious diarrhea, irritable bowel syndrome, small intestinal anal disease, Crohn's disease, and/or pancreatic insufficiency. Rifaximin is licensed by the U.S. Food and Drug Administration to treat traveler's diarrhea caused by E. coli.

Rifaximin has low systemic absorption with Cₘₐₓ of 3.4 ng/mL, Tₘₐₓ of 0.8 hours and is moderately bound to plasma proteins (67.5%). It has half-life of 1.8 hours and is primarily excreted in feces (97% of administered dose) and 0.32% in the urine.

Rifaximin is not absorbed by the oral route [Venturini A. P., Chemotherapy, 29, 1-3, (1983)] nor by topical application [Venturini A. P. et al., Drugs Exptl. Clin. Res., 13, 4, 233-6, (1987)].

Due to this particular pharmacokinetic behaviour, rifaximin has no toxicity at a dose of 2000 mg/kg/os, when administered orally in rats, and therefore, on the basis of the microbiological, pharmacodynamic and toxicological data, the drug has been used for the treatment of bacterial gastroenteritis, neurological symptoms and clinical symptoms of hepatic encephalopathy and for the pre- and post-surgical treatment of the gastrointestinal tract [Alvisi V. et al., J. Int. Med. Res., 15, 49-56, (1987), Testa R. et al., Drugs Exptl. Clin. Res., 11, 387-392, (1985), Gruttadauria G. et al., Eur. Rev. Med. Pharm. Sci., 9, 100-105, (1987)].

Rifaximin is used for the treatment of pathologies caused by non-invasive strains of Escherichia coli, a micro-organism which is not able to penetrate into GI mucosa and therefore remains in contact with gastrointestinal fluids.

Rifaximin is also approved for the treatment of pathologies whose etiology is in part or totally due to intestinal acute and chronic infections sustained by Gram-positive and Gram-negative bacteria, with diarrhea syndromes, altered intestinal microbial flora, summer diarrhea-like episodes, traveler's diarrhea and enterocolitis; pre- and post-surgery prophylaxis of the infective complications in gastro intestinal surgery; and hyperammonaemia therapy as coadjutant.

Rifaximin is available in tablets, granules for oral suspension and ointment, marketed in Europe and U.S.A. and in many other countries. Tablets, for example are currently marketed at the dosage of 200 mg for traveler's diarrhea under the brand name Xifaxan®.

Italian Patent 1,154, 655 and in U.S. Pat. No. 4,341,785 both discloses the compound 4-desoxy-4′-methyl-pyrido[1′2′:1.2] imidazo[5.4-c] rifamycin SV which is rifaximin.

U.S. Pat. No. 7,045,620 S.p.A. to Alfa Wassermann and WO2006/094662 both discloses polymorphic forms of rifaximin.

U.S. Pat. No. 4,341,785 to Marchi et al. discloses imidazo-rifamicyn derivatives having antibacterial utility, and the related process for preparing it. The patent also discloses a pharmaceutical antibacterial composition and a method of using it to treat antibacterial diseases of the gastrointestinal tract.

European Patent No. EP0161534 to Cannata et al. discloses a process for the synthesis of pyrido-imidazo rifamycins. The process is described as an improvement over the '785 patent to Marchi in that the later process provides unsatisfactory yields from an industrial point of view.

European Patent No. EP0858804 to Ferrieri et al. describes use of oral rifaximin compositions in the treatment of diarrhea from cryptosporidiosis.

U.S. Pat. No. 5,352,679 to Ferrieri et al. describes use of rifaximin (INN) in formulations for treatment of gastric dyspepsia caused by Helicobacter pylori bacteria. The rifaximin formulations disclosed in the patent are in the form of tablet, capsule, sugar coated tablet, granules or syrup for oral administration.

U.S. Pat. Nos. 5,314,904 and 6,140,355 both to Egidio et al. disclose compositions containing rifaximin for treatment of vaginal infections.

WO 2007/103448 discloses pharmaceutical preparations comprising an anti-rectal dysfunction agent and rifaximin. The preferred anti-rectal dysfunction is a nitric oxide modulating agent such as nitroglycerin. The examples disclosed in the patent application are related to the ointment containing rifaximin and nitroglycerine.

EP-A-0468555 and EP-A-0395329, by Smith Kline & French and FR-A2647344 by Physiopharm discloses aqueous foam compositions in which the same substance or mixture of substances (namely one or more chlorofluorocarbons) is used as both a foaming agent and a propellant for expulsion of the composition out of a conventional aerosol can.

However, there is little disclosure in the prior art about the topical formulations of rifaximin which are capable of providing the desired effect.

It is known that topical treatment of infections or disturbances of the colon or rectum is more preferred than oral route, as the formulation is directly applied to the site of action and hence rapidly reaches and acts on the point at which the disturbance is located.

According to the state of the art, topical delivery of active agents is achieved preferably by rectal administration using suppositories, enemas, ointments, creams and foam. Of these the suppository is the most common one. The suppository base is generally a fat but also water-soluble or water-miscible bases are utilized. To obtain a good bioavailability the active ingredient should come into contact with the rectal or colonic mucosa.

Ointments and creams often do not create an environment for promoting respiration of the wound tissue and it is not favorable to the normal respiration of the skin. Moreover, there may be likelihood of experiencing pain and irritation during the application of ointments and creams, particularly to abraded, wounded or inflamed mucosa of the rectum or colon.

Aqueous foamable preparations are the less common of the rectal preparation forms. They require a relatively complicated manufacture as well as complicated packaging compared with suppositories and enema. However, since better spreading effects are obtained with enema and foams than with suppositories more distal intestine regions can be reached thereby.

Although the delivery of active ingredient using foam can provide various advantages as compared to the other topical delivery forms such as better spreading in the surrounding tissues, rectal foams are complicated formulations which may not form under arbitrary circumstances because it requires a special balance between the foam-forming components. Slight shifts in the composition may result in a collapse of the foam or alternatively the foam is not formed at all, especially when administration is to occur via an applicator nozzle with small diameter. Most foam dosage forms for rectal delivery have incorporated corticosteroids to date, although some products have also been used to deliver antiseptics, antifungal agents, anti-inflammatory agents, local anesthetic agents, skin emollients, and protectants (American Journal of Drug Delivery, 2003, vol. 1(1), pp. 71-75). However, only a few are commercially available as yet.

Conventional foams for rectal or vaginal administration are filled in pressurized containers with a pharmaceutically active ingredient dissolved or suspended in a liquid vehicle, at least one propellant gas and a surfactant with foaming properties. Examples based on mesalazine, peppermint, sucralfate or budesonide as the active ingredient dispersed in a liquid vehicle containing a foaming surfactant and administered for topical action in the colon using a pressurised atomiser with a propellant gas are described in EP-A-468 555.

Because of the hydrophobic nature of rifaximin, it is virtually insoluble in water but is readily soluble in alcohols. An adequate amount of active substance can be dissolved by the use of solubilizers such as organic solvents, water-soluble alcohols. However the formulations if prepared in this way; may remain stable over a short period because large amounts of the active substance are decomposed within a short time.

Because of this problem, topical rifaximin formulations which can be used directly by the patient in the administration form ready for use have remained challenging. The suitable compositions of rifaximin suggested in the prior art are ointment and vaginal foam. The ointment is not in the form of ready to use, but can be prepared by a cumbersome process of crushing the rifaximin tablet in suitable oily vehicle and admixing this mixture with ointment base prior to the application. Moreover, the vaginal foam when formulated may also not remain stable when provided in compressed gas packs.

Thus, there exists a need to develop a topical foam composition of rifaximin suitable for rectal administration, useful to reduce healing time, alleviate pain and promote healing of the affected rectal and anal tissues which also remains stable over the storage period.

However, there are problems in producing an effective stable rifaximin composition, suitable for rectal administration, in either an aqueous and/or non-aqueous vehicle.

### Object

An object of the present invention is to provide a topical foam composition of rifaximin suitable for rectal administration. It is a particular object to provide effective formulations having either an aqueous and/or a non-aqueous vehicle.

Another object of the present invention is to provide a topical foam composition of rifaximin having better spreading effect.

Yet another object of the present invention is to provide a topical foam composition of rifaximin which remains stable over the storage period.

Yet another object of the present invention is to provide a method of manufacturing the topical pharmaceutical composition of rifaximin suitable for rectal administration.

Still another object of the present invention is to provide is to provide a method of treating, prophylaxis, or maintenance of remission of colonic or rectal dysfunction by administering the topical foam composition of rifaximin to patients in need thereof.

A further another object of the present invention is to provide a topical foam composition of rifaximin for rectal administration which remains effective even after intestinal evacuation by the subject treated.

### SUMMARY

According to the first aspect of the present invention there is provided a topical foam composition of rifaximin for rectal administration.

According to a further aspect there is provided a topical foam composition of rifaximin for rectal administration comprising one or more pharmaceutical excipients or carriers such as at least one surfactant and at least one propellant or mixtures thereof.

According to a further aspect there is provided a process of manufacturing the said topical foam composition of rifaximin.

According to a further aspect there is provided a topical foam composition of rifaximin for use in the preparation of a medicament suitable for administering to the rectum, colon and/or terminal ileum of a patient for the treatment, prophylaxis, or maintenance of remission of colonic or rectal dysfunction.

According to yet another aspect there is provided a method of treating, preventing, or alleviating an anal disorder comprising administering the topical foam of rifaximin to a subject in need thereof.

### DETAILED DESCRIPTION

As discussed above, there is a need to develop a topical foam composition of rifaximin suitable for rectal administration.

The present inventors have developed a topical foam composition of rifaximin which may achieve the aforesaid objectives and which also exhibit a topical anti-infective action.

Surprisingly, the present inventors have found that by utilizing appropriate combination of pharmaceutical excipients or carriers it is possible to achieve a topical pharmaceutical composition of rifaximin suitable for rectal administration and achieve the aforesaid objectives. Specifically, the inventor shave found a way to formulate rifaximin as a topical foam composition by means of either an aqueous and/or non-aqueous vehicle.

In particular, by use of a specific ratio of water soluble alkanols to water in topical foam composition comprising rifaximin, it remains stable over the storage period. The ratio of water soluble alkanol to water is preferably from about 0.05:10 to 10:0.05, on a weight basis.

Pharmaceutically acceptable water soluble alkanols which are suitable for use in the present invention may be selected from, but not limited to ethanol, polyalcohols such as a propylene glycol, glycerol, polyethyleneglycol, polypropylene glycol, propylene glycol glyceryl esters or mixtures thereof.

As used herein, the term ‘rifaximin’ is used throughout the description in broad sense to include not only rifaximin per se but also its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. Rifaximin used may also be in Polymorphic form or amorphous form. Examples of polymorhic forms of rifaximin include, but not limited to polymorph [alpha], polymorph [beta], polymorph [gamma], polymorph [delta], and polymorph e of rifaximin, as described in U.S. patent application Ser. No. 10/728,090, U.S. patent application Ser. No. 11/135,651, European Patent Application No. 04005541 and European Patent Application No. 15227.

The present invention provides a topical foam composition comprising rifaximin suitable for rectal administration in with one or more pharmaceutically acceptable excipients or carriers and which also remains stable over the storage period.

The amount of rifaximin in the rectal foam composition according to the present invention preferably ranges from about 0.01% w/w to about 10% w/w, more preferably from about 0.5% w/w to about 8% w/w relative to the total weight of the composition.

In an embodiment, the present invention provides a topical foam composition comprising rifaximin with one or more pharmaceutical excipient/carrier in the form of a rectal foam filled in a compressed gas container, that upon valve actuation, emits a fine dispersion of liquid and/or solid materials in a gaseous medium. The said composition is easier to apply, less dense, and spread more easily than other topical dosage forms. Alternatively, the composition may be formulated in various ways to provide emollient or drying functions to the rectal mucosa, depending on the formulation constituents. Another benefit of the pharmaceutical composition of the present invention is ease of use by the patient and consumer acceptance.

In a further embodiment, the topical foam composition may not contain mineral oils. The disadvantage of these components is that they might further irritate the already inflamed areas of the rectum or anus.

In a preferred embodiment, the topical foam composition of the present invention comprises rifaximin, at least one surfactant and at least one propellant, water soluble alkanols, water and optionally other pharmaceutical excipients or carriers.

Although a mixture of water soluble alkanols is the preferred vehicle for the topical foam composition according to the present invention, suitable non-aqueous vehicle which may be employed in the topical foam composition of the invention, which include but not limited to vegetable oils, such as olive oil; injectable organic esters, such as ethyl oleate or mixtures thereof.

It is also desirable to use a suitable vehicle which is compatible with the rectal and colonic mucosa.

Alternatively, the liquid vehicle may also be based on highly hydrophilic organic substances to allow the surfactant to perform its foaming action, which however preferably should not be inhibited by the other substances present in the formulation, such as the active principles and their stabilizers, whereas the specific adjuvants (such as foam consistency correctors) are preferably chosen from those with strong hydrophilic and lipophilic characteristics.

The vehicle typically constitutes from 10% w/w to 95% w/w, preferably from 10% w/w to 90% w/w, more preferably from 20% to 70% w/w relative to the total weight of the composition.

In a preferred embodiment, the vehicle employed in the topical foam composition of the present invention comprises water in an amount from approximately 20% w/w to approximately 90% w/w relative to the total weight of the composition and a water-soluble alkanol, preferably propylene glycol, in an amount from approximately 0% w/w to 50% w/w relative to the total weight of the composition. Preferably, the vehicle contains 20-80% w/w water relative to the total weight of the composition. Preferably the vehicle contains 5-40% w/w water soluble alkanol relative to the total weight of the composition. Most preferably, the vehicle contains 20-80% w/w water relative to the total weight of the composition, and 5-40% w/w water soluble alkanol relative to the total weight of the composition.

The preferred amount of non-aqueous vehicle, especially the water soluble alkanol, more especially the propylene glycol, is from 10% to 40% w/w based on the total weight of the composition.

Examples of surface active agents which may be employed in the aqueous foam composition of the present invention include, but not limited to anionic surfactants, non-ionic surfactants, cationic surfactants, and amphoteric surfactants. Examples of anionic surfactants include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinate, disodium lauryl sulfosuccinate, diammonium lauryl sulfosuccinate, tetrasodium N-(1,2-dicarboxyethyl)-N-octadecylsulfosuccinate, diamyl ester of sodium sulfosuccinic acid, dihexyl ester of sodium sulfosuccinic acid, dioctyl esters of sodium sulfosuccinic acid, docusate sodium, and combinations thereof. The preferred amount of surfactant, is present in an amount from 0.1% to 10.0% w/w based on the total weight of the composition; more preferably, in an amount from 0.1% to 8.0% w/w based on the total weight of the composition.

Examples of nonionic surfactants include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethylene oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof.

Examples of amphoteric surfactants include, but are not limited to, sodium N-dodecyl-1-alanine, sodium N-lauryl-1-iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and combinations thereof.

Examples of cationic surfactants include, but are not limited to, behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowedimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquatemium, stearalkonium chloride, sterayl dimethylbenzyl ammonium chloride, stearyl trimethyl ammonium chloride, trimethylglycine, and combinations thereof.

It will be appreciated by the person skilled in the art that amongst the two or more surfactants selected, at least one surfactant selected may provide the emulsifying action whereas the other may provide a foam-stabilizing action. It will also be appreciated that at least one surfactant employed may possess emulsifying action and another surfactant employed may possess foam stabilizing action. The surfactant(s) is desirably chosen such that it remains compatible with the rectal and colonic mucosa and will be present in an amount which achieves the desired pharmaceutical effect but which does not give rise to problems of irritation.

In a further embodiment of the present invention, the topical foam composition contains a lubricant. Preferably, said lubricant is a silicone (e.g. polydimethylsiloxane). The silicone may further stabilize the foam-forming composition.

The propellant used in the topical foam composition of the present invention is used to accomplish the foaming effect. The propellant may be chosen according to known principles for preparing a foamable composition of the aerosol type packed in a pressurized container and suitable for a rectal application. The propellant may be any suitable, pharmaceutically acceptable, gas such as a low molecular weight hydrocarbon e.g. isobutane, n-butane, propane, CFC, hydrocarbons; chlorofluorocarbons (CFCs); hydrochlorofluorocarbons (HCFCs); hydrofluoroalkanes (HFAs) such as HFA 134a and HFA 227; or air. Preferably, the propellant comprises a mixture of n-butane, isobutane, propane.

The propelling properties can vary depending on the type and quantity of propellant used and, consequently, the foam can reach more or less distant regions of the large intestine.

The propellant may be present in an amount from 0.05 to 20% w/w, preferably 0.5 to 20% w/w, of the composition. Preferably, said amount is between 3 to 10%, more preferably between 7 to 9% w/w of the composition. Additionally, liquefied nitrogen may be present as pressurizing agent to obtain the required number of doses.

Further, the topical foam composition according to the present invention may comprise at least one additional active ingredient suitable for rectal administration.

Additional active agents may be may be selected from, but not limited to one or more of anti-inflammatory agents, steroids (e.g. corticosteroids), additional antibiotics, anti-fungal agents, analgesics, or anti-neoplastic agents.

Examples of suitable antibiotics includes, but not limited to: dapsone, chloramphenicol, neomycin, cefaclor, cefadroxil, cephalexin, cephradine, erythromycin, clindamycin, lincomycin, amoxicillin, ampicillin, bacampicillin, carbenicillin, diel oxacillin, cyclacillin, picloxacillin, hetacillin, methicillin, nafcillin, penicillin, polymyxin, tetracycline, amphotericin-b, candicidin, dermostatin, filipin, fungichromin, hachimycin, hamycin, lucensomycin, mepartricin, natamycin, nystatin, pecilocin, perimycin, azaserine, griseofulvin, oligomycins, neomycin undecylenate, pyrroinitrin, siccanin, tubercidin, viridin, picloxacillin, hetacillin, methicillin, nafcillin, penicillin, polymyxin, or tetracycline.

Examples of suitable anitfungal agents includes but not limited to: allylamines such as butenafine, naftifine, imidazoles such as bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole, triazoles such as fluconazole, itraconazole, saperconazole, terconazole, and others such as acrisorcin, amorolfliotalne, biphenamine, bromosalicylchloranilide, buclosamide, calcium propionate, chlophenesin, ciclopirox, cloxyquin, coparaffliotalnate, diamthazole, dihydrochloride, exalamide, flucytosine, halethazole, hexetidine, Ioflucarban, nifuratel, potassium iodide, propionates, propionic acid, pyrithione, salicylanilide, sulbentine, tenonitrozole, triacetin, ujothion, undecylenic acid.

Antifungal agents may also include, for example, polyenes such as amphotericin-b, candicidin, dermostatin, filipin, fungichromin, hachimycin, hamycin, lucensomycin, mepartricin, natamycin, nystatin, pecilocin, perimycin, azaserine, griseofulvin, oligomycins, neomycin undecylenate, pyrroinitrin, siccanin, tubercidin, viridin, allylamines such as butenafine, naftifine, imidazoles such as bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole, triazoles such as fluconazole, itraconazole, saperconazole, terconazole, acrisorcin, amorolfliotalne, biphenamine, bromosalicylchloranilide, buclosamide, calcium propionate, chlophenesin, ciclopirox, cloxyquin, coparaffliotalnate, diamthazole, dihydrochloride, exalamide, flucytosine, halethazole, hexetidine, Ioflucarban, nifuratel, potassium iodide, propionates, propionic acid, pyrithione, salicylanilide, sulbentine, tenonitrozole, triacetin, ujothion, or undecylenic acid.

The other therapeutic agent can include steroid or a non-steroidal antiinflammatory agent. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflum[iota]c acid, tolfenamic acid, diflurisal, flufenisal, piroxicam, sudoxicarn, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof.

Examples of suitable corticosteroid includes but not limited to: hydrocortisone, i.e., 11-17-21-trihydroxypregn-4-ene-3,20-dione or Cortisol, Cortisol acetate, hydrocortisone phosphate, hydrocortisone 21-sodium succinate, hydrocortisone tebutate, corticosterone, corticosterone acetate, cortisone, cortisone acetate, cortisone 21B-cyclopentanepropionate, cortisone phosphate, triamcinolone hexacetonide, dexamethasone phosphate, desonide, betamethasone dipropionate, mometasone furate.

The corticosteroid and topical anesthetic may be employed together in the composition along with rifaximin

For inflammation, preferred treatments for use in combination therapy with the compositions of the present invention include, but not limited to: naproxen sodium (Anaprox(R) and Anaprox(R) DS, Roche), flurbiprofen (Ansaid(R); Pharmacia), diclofenac sodium+misoprostil (Arthrotec(R), Searle), valdecoxib (Bextra(R), Pharmacia), diclofenac potassium (Cataflam(R) and Voltaren(R), Novartis), celecoxib (Celebrex(R), Pfizer), sulindac (Clinoril(R), Merck), oxaprozin (Daypro(R), Pharmacia), salsalate (Disalcid(R), 3M), difhmisal (Dolobid(R), Merck), naproxen sodium (EC Naprosyn(R), Roche), piroxicam (Feldene(R), Pfizer), indomethacin (Indocin(R) and Indocin SR(R), Merck), etodolac (Lodine(R) and Lodine XL(R), Wyeth), meloxicam (Mobic(R), Boehringer Ingelheim), ibuprofen (Motrin(R), Pharmacia), naproxen (Naprelan(R), Elan), naproxen (Naprosyn(R), Roche), ketoprofen (Orudis(R) and Oruvail(R), Wyeth), nabumetone (Relafen(R), SmithKline), tolmetin sodium (Tolectin(R), McNeil), choline magnesium trisalicylate (Trilisate(R), Purdue Fredrick), and rofecoxib (Vioxx(R), Merck).

Antineoplastic agents may also be included in the topical foam composition of the present invention along with the rifaximin include, but not limited to: vincristine, vinblastine, vindesine, busulfan, chlorambucil, spiroplatin, cisplatin, carboplatin, methotrexate, adriamycin, mitomycin, bleomycin, cytosi[pi]e arabinoside, arabinosyl adenine, mercaptopurine, mitotane, procarbazine, dactinomycin (antinomycin D), daunorubicin, doxorubicin hydrochloride, taxol, plicamycin, aminoglutethimide, estramustine, flutamide, leuprolide, megestrol acetate, tamoxifen, testolactone, trilostane, amsacrine (m-AMSA), asparaginase (L-asparaginase), etoposide, and interferon a-2a and 2b.

Antiviral agents may also be included in the topical foam composition of the present invention along with the rifaximin include, but not limited to: acyclovir, amantadine, azidothymidine, ribavirin or vidarabine. In any case where pain in a component of the target disorder, the other therapeutic agent can be an analgesic. Useful analgesics include, but are not limited to, phenacetin, butacetin, acetaminophen, nefopam, acetoamidoquinone, and mixtures thereof.

Optionally, a topical anesthetic may be present in the composition of the invention. For instance, the topical anesthetic may include, but not limited to dibucaine, lidocaine, pramoxine, benzocaine, tetracaine. In general, the topical anesthetic may be present in any amount which is effective in the practice of the treatment of anal disease.

In a preferred embodiment, the present invention relates to a pharmaceutical combination product comprising rifaximin adapted for delivery to the colon and/or rectum and a compound selected from, but not limited, one or more of 5-acetyl salicylic acid (5-ASA), sulphasalazine, asalazine, prednisolone, or budesonide for simultaneous, separate, or sequential administration.

The topical foam composition according to the present invention is usually packed in a suitable pressurized dispensing canister of the aerosol type well known in the art such as an aluminium canister. Each canister is sealed with a suitable foam dispensing valve. Any valve or nozzle/valve assembly which provides a means for releasing the foam from the container and provides foam which is suitable for use in the present invention may be used. The foam that is formed from the composition of the present invention has superior properties. The advantages associated with the topical foam composition according to the present invention is that better results may be obtained in combating the disease and either a lower dosage of the active ingredient or less dosages per day may be necessary to obtain similar results when compared with prior art compositions. For instance, the increased spreading of the foam together with the longer exposure time to the active will result in optimal local effect at the target site. Also, the foam of the present invention may not cause extra irritation of the inflamed target mucosa due to the absence of mineral oils as present in the prior art compositions. Due to these superior properties of the foam, the current invention may represent a valuable alternative to previously known medicines used for the treatment of rectal diseases.

The topical foam composition of the present invention is presented in a suitable dispensing container, for example an aluminium aerosol container, fitted with a suitable metered or un-metered valve. Such containers are well known in the art. Where desired, the container can be fitted or supplied together with an applicator device for insertion into the rectum to ensure more efficient administration of the foam.

The dispensing container may be in the form of coated aluminium cans to prevent corrosion, such as epoxy-coated cans. At the time of application, the mixture of the mixing of the ingredients with propellant may be insured by shaking, optionally with the aid of a mixing bead. The can may be arranged for either “upside down” spraying with the valve at the bottom, or the can have a dip tube so that the foam can be sprayed while the can is upright with the valve at the top.

During the use, the dispensing valve of the can allows rapid expansion of the propellant, which triggers and enhances the foaming action of the surfactant, which thus emerges to entrain the medicated liquid in the form of foam.

The propellant expansion energy is absorbed mainly in forming the foam, thus allowing rectal application without risk.

According to the present invention, the foam may be generated at the moment of therapeutical application. Hence the known formulation and dispensing technology used in the state of the art applicable to foam cans, for example in cosmetics is therefore suitable. Preferably, the active agent is vehicled in the liquid state with at least one propellant and a surfactant with foaming action.

The topical foam composition of the present invention is applied proximate or to the affected area of the external anus or distal anal canal of the subject.

On administering such compositions, it is sufficient to obtain foams of medium consistency, with a minimum volume of 0.5 g to 10 g of foam introduced into the rectum.

The present invention further provides a process of manufacturing the topical foam composition of rifaximin comprising:

(1) Heating the mixture of emulsifying wax, emulsifier with surfactant and preservative-water separately.
(2) Adding water soluble alkanol to the preservative-water solution and then mixing with oily phase of step (1)
(3) Adding rifaximin to the above mixture under stirring and adjust the required pH using suitable pH adjusting agent.
(4) Make up the volume of the mixture by addition of purified water and finally fill the blend in metal cans and charge the can with propellant.

In a preferred embodiment, rifaximin is used in micronized form, preferably of the size less that about 200 microns. More preferably, the size may be less than about 150 microns or less.

The active agent is solubilized or suspended in a suitable liquid vehicle containing a foaming surfactant. The liquid is placed in an atomizer can sealed by a dispensing valve and then pressurized by feeding a suitable quantity of propellant through the valve.

The present composition can be prepared by mixing the ingredients in an appropriate manner and then filling into a suitable dispensing container, for example as described in the examples.

It will be appreciated by the person skilled in the art that the topical foam composition comprising rifaximin further may comprise one or more pharmaceutical excipients, selected from, but are not limited to: emollient or humectants, pH adjusting agent, emulsifiers, foaming agents, fatty alcohol, preservative, chelating agents, antioxidants, suspending agents, thickening agents, permeation enhancers, occlusive agents, colorants and fragrances or combinations thereof.

Examples of suitable pH adjusting agents may be selected from, but not limited to, sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof, preferably triethanolamine is used.

In a preferred embodiment, the topical foam composition according to the present invention topical comprises suitable pH adjusting agent to adjust the pH in the range from approximately 4 to 8.

Examples of the emulsifying waxes that can be used in the topical foam composition of the present invention are non-ionic emulsifying waxes such as those described in the U.S. National Formulary (USNF) and ‘Martindale’. An emulsifying wax may be incorporated in the topical composition of the present invention in order to stiffen the foam. The amount of emulsifying wax in the composition is preferably from 1% to 10% w/w based on the total weight of the composition.

Examples of surfactants which may be employed in the topical foam composition of the present invention include, but not limited to fatty alcohol for example, cetyl stearyl, lauryl, myristyl, and palmityl alcohols, surfactants or mixtures thereof. A preferred surfactant is polyoxyethylene 10 stearyl ether. and, is preferably present in an amount from 0.1% to 1.0% w/w based on the total weight of the composition.

In another embodiment according to the present invention, a suitable surface active agent can be employed which performs the function of both foaming agent and surfactant.

Examples of suitable emollients and/or humectants which may be employed in the topical foam composition of the present invention include, but not limited to, polyhydric alcohols such as glycols, and polysaccharides, such as ethylene glycol, propylene glycol, butylene glycol, diethylene glycol, dipropylene glycol, glycerin, diglycerin, sorbitol, malvitol, trehalose, raffinose, xylitol, mannitol, polyethylene glycol, propylene glycol, polyglycerin, cholesterol, squaline, fatty acids, octyldodecanol, myristyl alcohol, urea, lanolin, lactic acid, esters such as isopropyl stearate, isopropyl myristate, isopropyl palmitate and isopropyl laurate and the like, preferably myristyl alcohol, octyldodecanol, propylene glycol.

In a preferred embodiment for mucosal delivery, permeation enhancers may be incorporated in the topical foam composition of the present invention for delivery of the active ingredient to the mucosal surface. Most types of enhancers are detergents that include: sodium glycocholate, sodium taurocholate, polysorbate 80, sodium lauryl sulfate, lauric acid, and various alkyl glycosides. Other examples of enhancers include: dextrins (cyclodextrin, dextran sulfate), fatty acids (phosphatidylcholine, lysophosphatidylcholine), heterocyclic compounds (azone), and small molecules (benzalkonium chloride, cetyltrimethylammonium bromide). Each is contemplated for use in the present invention as are other unlisted ingredients typically used for such purpose, as would be appreciated by one of skill in the art.

In another preferred embodiment, suitable mucoadhesives may be used in the aqueous foam composition of the present invention to improve local retention of mucosally delivered of the active ingredient.

Mucoadhesive compounds are primarily synthetic or natural polymers that can adhere to the wet mucosal surface. These include synthetic polymers such as, but not limited to monomeric alpha cyanoacrylate, polyacrylic acid, hydroxypropyl methylcellulose, and poly methacrylate derivatives. Glue-like polymers include epoxy resins and polyurethanes. Naturally occurring mucoadhesives include chitosan, hyaluronic acid and xanthan gum or mixtures thereof.

Suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof. One preferred emulsifier is cetyl alcohol. The emulsifier, for example the cetyl alcohol is preferably present in an amount from 0.1 to 5.0% w/w based on the total weight of the composition.

Suitable suspending agents include, but are not limited to, alginic acid, bentonite, carbomer, carboxymethylcellulose and salts thereof, colloidal oatmeal, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof. Suitable chelating agents include, but are not limited to, EDTA, disodium edetate, trans-1,2-diaminocyclohexane-N,N,N′,N′-tetraacetic acid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosphonic acid), 0,0′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphonic acid), 7,19,30-trioxa-1,4,10,13,16,22,27,33-octaazabicyclo[111,11,1]pentatriacon-tane hexahydrobromide, triethylenetetramine-N,N,N′,N″,N′“,N′”-hexaacetic acid, and combinations thereof.

Suitable emollients include, but are not limited to, myristyl lactate, isopropyl palmitate, light liquid paraffin, cetearyl alcohol, lanolin, lanolin derivatives, mineral oil, petrolatum, cetyl esters wax, cholesterol, glycerol, glycerol monostearate, isopropyl myristate, lecithin, and combinations thereof.

Preservatives can be used to prevent the growth of fungi and other microorganisms. Suitable preservatives include, but are not limited to, benzoic acid, sorbic acid, butylparaben, ethyl paraben, methyl paraben, propyl paraben, sodium benzoate, sodium propionate, benzalkonium chloride, benzethonium chloride, benzyl alcohol, cetylpyridinium chloride, chlorobutanol, phenol, phenylethyl alcohol, thimerosal, and combinations thereof. The preservative is preferably present in an amount from 0.01% to 0.20% w/w, preferably 0.1% to 0.20% w/w, based on the total weight of the composition. In a particular embodiment the composition contains 0.1% to 0.18% w/w methyl paraben and 0.01% to 0.02% w/w propyl paraben.

Examples of suitable antioxidant include, but not limited to sodium metabisulphite and advantageously this can be used in conjunction with a chelating agent such as a salt of EDTA, e.g. disodium edetate.

The present invention further provides a method of treating, preventing, or alleviating anal disorders comprising administering to a subject in need thereof an effective amount of rifaximin. Anal disorders include one or more of anal fissure, anal ulcer, haemorrhoidal disease, levator spasm, inflammatory bowel disease with anal involvement, irritable bowel syndrome, diarrhea, microbe associated diarrhea, Clostridium difficile associated diarrhea, travelers' diarrhea, small intestinal anal disease, Crohn's disease, chronic pancreatitis, pancreatic insufficiency, colitis, hepatic encephalopathy, or pouchitis. In treatment according to the invention, the composition of the invention is contacted with or applied to the affected anal area or proximate thereto such that an effective amount of active ingredient is administered.

In a preferred embodiment, the amount of composition which is employed should be effective for the amelioration, control and/or healing of the anal disease and the prompt and dramatic control or relief of pain resulting from or associated with the disease.

According to yet another embodiment, the anal disorder is or is caused by one or more of anal fissure, anal ulcer, and acute haemorrhoidal disease, irritable bowel syndrome, inflammatory bowel disease, (e.g., Crohn's and colitis), travelers' diarrhea, large intestinal anal disease, chronic pancreatitis, pancreatic insufficiency or post-surgical disease (e.g., pouchitis).

In a further embodiment, the effective amount is effective to treat a bacterial infection, e.g., anal diseases including, one or more of anal fissure, anal ulcer, and acute haemorrhoidal disease, irritable bowel syndrome, travelers' diarrhea, small intestinal anal disease, Crohn's disease, chronic pancreatitis, pancreatic insufficiency, colitis, hepatic encephalopathy, antibiotic associated colitis, and/or diverticular disease.

The following examples are for the purpose of illustration of the invention only and are not intended in any way to limit the scope of the present invention.

### Examples

### Example 1

| | | Qty/Unit |
|---|---|---|
| Sr. No. | Ingredients | (% w/w) |
| 1 | Rifaximin | 5 |
| 2 | Propylene glycol | 20.00 |
| 3 | Emulsifying wax | 1.50 |
| 4 | Cetyl alcohol | 0.18 |
| 5 | Polyoxyethylene 10 stearyl ether | 0.25 |
| 6 | Methyl hydroxybenzoate or Methyl paraben | 0.10 |
| 7 | Propyl hydroxybenzoate or propyl paraben | 0.01 |
| 8 | Triethanolamine | q.s. to pH 6.0 |
| 9 | Purified water | q.s. to 100 g |
| 10 | Propellant (Propane/n-Butane/Isobutane) | 4.00 g |
| | Total | 104.00 g |

### Process:

(1) Heat the emulsifying wax, cetyl alcohol and polyoxyethylene 10 stearyl ether.
(2) Heat methyl paraben or methyl hydroxybenzoate and propyl paraben or propyl hydroxybenzoate with water.
(3) Add propylene glycol to the solution of step (2) under homogenization.
(4) Add mixture of step (1) to the solution of step (3) under homogenization and allow to cool under stirring.
(5) Add rifaximin (micronized, less than 200 microns) to the above mixture and homogenize to cool at room temperature.
(6) A solution of triethanolamine is added to the above mixture to adjust the pH about 6.
(7) Make up the volume of the mixture by addition of purified water
(8) Finally fill the blend in metal cans and charge the can with propellant.

### Example 2

### Non Aqueous Foam

| Sr. | | Qty/unit |
|---|---|---|
| No | Ingredients | (% w/w) |
| 1. | Rifaximin | 5.00 |
| 2. | Cetostearyl Alcohol | 2.00-8.00 |
| 3. | Triglycerides of capric/caprylic acid | 80.00-95.00 |
| 4. | Propyl paraben | 0.01-0.02 |
| 5. | Butylated hydroxytoluene (BHT) | 0.01-0.1 |
| 6. | Propane/n-butane/iso-butane | 2.00-10.00 |

### Process:

1. Heat part quantity of Triglycerides of capric/caprylic acid, BHT, Propyl paraben and cetostearyl alcohol to about 60-70° C.
2. Homogenize the above mixture for 10 minutes and allow to cool.
3.Separately, heat part quantity of Triglycerides of capric/caprylic acid and rifaximin and homogenize for 10 minutes.
4. Add the above mixture step (3) in the mixture obtained in step (2) maintained at 45° C. under stirring.
5. Cool to room temperature under stirring and fill the prepared blend in aluminium canisters and seal with dispensing valves
6. Charge specified amount of propellant through these valves.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “cosolvent” refers to a single cosolvent or to combinations of two or more cosolvents, and the like.

1. A pharmaceutical composition for topical rectal administration in the form of a foam, the composition comprising rifaximin.

2. A composition according to claim 1, further comprising an aqueous or non-aqueous vehicle.

3. A composition according to claim 2, comprising wherein the non-aqueous vehicle is one or more pharmaceutically acceptably alkanols; one or more pharmaceutically acceptable vegetable oils; or one or more pharmaceutically acceptable organic esters.

4. A composition according to claim 3, wherein the water soluble alkanol is ethanol; propylene glycol; glycerol; polyethyleneglycol; polypropylene glycol; propylene glycol; a glyceryl ester; or a mixture thereof.

5. A composition according to claim 2, wherein the vehicle comprises a water soluble alkanol and water, and wherein the w/w ratio of the water soluble alkanol to water from 0.05:10 to 10:0.05.

6. A composition according to claim 2, wherein the vehicle constitutes from 10% w/w to 90% w/w of the total weight of the composition.

7. A composition according to claim 2, wherein the vehicle comprises water in an amount from 20% w/w to 90% w/w of the total weight of the composition, and a water-soluble alkanol in an amount from 0% w/w to 50% w/w of the total weight of the composition.

8. A composition according to claim 1, further comprising at least one surfactant.

9. A composition according to claim 8, wherein the surfactant is present in an amount of from 0.1 to 1.0 w/w of the total weight of the composition.

10. A composition according to claim 1, further comprising at least one propellant.

11. A composition according to claim 10, wherein the propellant is present in an amount from 0.5 to 20% w/w of the total weight of the composition.

12. A composition according to claim 1, further comprising at least one solubilizer.

13. A composition according to claim 1, further comprising at least one emulsifier.

14. A composition according to claim 13, wherein the emulsifier is present in an amount from 1% to 15% w/w of the total weight of the composition.

15. A composition according to claim 1, further comprising at least one antioxidant.

16. A composition according to claim 1, further comprising at least one preservative.

17. A composition according to claim 16, wherein the preservative is present in an amount from 0.1% to 0.2% w/w of the total weight of the composition.

18. A composition according to claim 1, further comprising a silicone.

19. A composition according to claims 1, comprising from 0.01% to 10% w/w rifaximin of the total weight of the composition.

20. A composition according to claim 1, which does not contain any mineral oil.

21. A composition according to claim 1, wherein the composition further comprises 5-acetyl salicylic acid (5-ASA), sulphasalazine, asalazine, prednisolone, or budesonide.

22. A pharmaceutical composition for topical rectal administration in the form of a foam, the composition comprising the following components in w/w:

| | |
|---|---|
| Rifaximin | 0.01-10% |
| Propylene Glycol | 10.0-40.0% |
| Emulsifying Wax | 1.0-10.0% |
| Cetyl Alcohol | 0.1-5.0% |
| Polyoxyethylene 10 | 0.1-1.0% |
| Stearyl Ether | |
| Methyl Paraben | 0.1-0.18% |
| Propyl Paraben | 0.01-0.02% |
| Triethanolamine | 0.5-5.0% |
| Purified Water | 20.0-80.0% |
| Hydrocarbon | 0.5-10.0% |
| Propellant | |
| (Propane/n-Butane/ | |
| Isobutane) | |

23. A pharmaceutical composition as defined in claim 22 for administration to the rectum, colon and/or terminal ileum of a patient for the treatment, prophylaxis, or maintenance of remission of a disorder of the rectum, colon, terminal ileum or anus.

24. The use of a pharmaceutical composition as defined in claim 22 in the manufacture of a medicament for administration to the rectum, colon and/or terminal ileum of a patient for the treatment, prophylaxis, or maintenance of a disorder of the rectum, colon, terminal ileum or anus.

25. A method of treating, preventing, or alleviating a disorder of the rectum, colon, terminal ileum or anus, comprising administering an effective amount of a pharmaceutical composition according to claim 22 to a subject in need thereof.

26. A process for manufacturing a pharmaceutical composition containing rifaximin, comprising:
(1) heating a mixture of emulsifier and surfactant to form an oily phase; (2) separately heating a mixture of a preservative and water;
(3) adding a water soluble alkanol to the preservative-water mixture and then mixing with oily phase of step (1); and
(4) adding rifaximin to the mixture formed in step (3) and adjusting the pH to a desired value using a pH adjusting agent.

27. A process according to claim 26, further comprising heating an emulsifying wax with the emulsifier and surfactant in step (1).

28. A process according to claim 26, further comprising optionally adding purified water to the product of step (4), then filling the product into a dispenser and charging the dispenser with a propellant.

29. A process according to claim 26, wherein the rifaximin is in micronized form.

30. A process according to claim 26, wherein the particle size of the rifaximin is less than about 200 microns.

31. The use of a silicone as a lubricant and a stabiliser in a rectal form composition containing rifaximin.

32. A dispenser for a pharmaceutical composition as defined in claim 22, comprising a canister containing under pressure the pharmaceutical composition; a metering valve for measuring a metered dose of the composition from the canister for administration to a patient in need thereof; and an actuator for actuating discharge of the metered dose of the formulation to patient in the form of a foam.

33. A dispenser according to claim 32, wherein the metered dose comprises 0.5 g to 10 g of the pharmaceutical composition.
